(19) United States
(12) Patent Application Publication
(10) Pub. No.: US 2008/0121305 A1
(43) Pub. Date: May 29, 2008

(54) Low friction fabric

(76) Inventor: Michael B. Metzger, Owings Mills, MD (US)

Correspondence Address: JOHN S. HALE;GIPPLE & HALE, 6665-A OLD DOMINION DRIVE, MCLEAN, VA, 22101, US

(21) Appl. No.: 11/907,478

(22) Filed: Oct. 12, 2007

Related U.S. Application Data
(63) Continuation-in-part of application No. 10/001,764, filed on Oct. 31, 2001, now Pat. No. 7,281,549.

(51) Int. Cl. D03D 15/10; A43B 17/00; D03D 25/00; A61F 13/00; A43B 19/00; A47G 9/02; A47C 21/00

(52) U.S. Cl. 139/421; 2/239; 139/383 B; 602/44; 36/43; 5/490; 5/482; 5/495

(57) ABSTRACT

A low friction fabric constructed of a first layer of woven polyester fibers with an upper and lower woven surface attached to and adjacent a second layer of the same weave of polyester or similar fibers, the second layer having an upper and lower surface. Each of the woven layers comprising a straight yarn in the warp of the weave pattern with the weaves of the layers being oriented at a 90 degree angle to one another.

### RELATED APPLICATIONS

This application is a Continuation In Part of application Ser. No. 10/001,764 filed Jul. 13, 2001, issuing as U.S. Pat. No. 7,281,549 on Oct. 16, 2007.

### STATEMENT REGARDING FEDERALLY SPONSORED RESEARCH OR DEVELOPMENT

None

### REFERENCE TO SEQUENCE LISTING, A TABLE OR A COMPUTER PROGRAM LISTING COMPACT DISC APPENDIX

None.

### BACKGROUND OF THE INVENTION

1. Field of Invention

This invention relates to a fabric designed to minimize shear forces. It has both medical and recreational applications.

2. Background of the Invention

The formation of calluses is primarily a result of friction. As the layers of skin are loaded in a shearing fashion, the planes of skin separate, leading to blistering in the space between layers. With further progression of shear loads, the upper layer or layers of skin can be traumatized to the point where it separates, leaving a painful, raw, exposed dermis. In addition to the pain associated with dermis exposure, there is a danger of progression of the sore as successive layers are forcefully torn away. Ultimately, this can lead to ulcerations of the skin that occur when the depth of the wound has advanced through the epidermis, dermis, and into the subcutaneous fat layer. The subcutaneous layer is highly vascular, and susceptible to infection.

This destructive process is a result of mechanical forces. In particular, the skin structure can be traumatized by vertical forces, perpendicular to the skin, or by shear forces, in the same plane as the skin. These excessive shear forces are the primary mechanical cause of various skin pathologies and a contributing factor to the failure of medical treatment modalities such as skin grafts. For many people excessive shear force is the primary cause of blistering during day-to-day activities and in those high impact activities that occur in many sports. An interface that is capable of reducing or eliminating shear forces would greatly reduce the potential for formation of blisters, and reduce the risk of subsequent ulcerations and infection. This is particularly a problem in many medical conditions where the patient has reduced sensitivity as a result of disease or medical procedure. In fact, the leading cause of non-traumatic amputation of a leg or foot is infection following ulcer formation in diabetic patients with neuropathy. In the US alone, nearly 60,000 amputations are performed annually due to non-healing ulcers, with an annual cost in excess of $2 billion, not to mention the physical debilitation and psychological trauma endured by the patient.

Previous attempts to reduce the shear force have utilized various polymers in the form of dimensional foams or gels. Rather than being transmitted to the user, shear forces are absorbed in the material itself. The compression and rotation of the material took up the shearing forces within the material instead of at the material skin interface.

The problem with this type of construction is that the amount of reduction in the shear is dependent on the property of the material itself, as well as its thickness. The thicker the material, the greater the reduction in shear forces. To provide adequate amounts of shearing between surfaces of the material there must be a nominal dimensional thickness to the foam or gel. As the material gets thinner less motion between surface layers occurs, thereby limiting its usefulness in reducing shear forces. Hence, the ability of dimensional polymers to reduce friction is dependent on their thickness and their unique chemical make up. How much side to side motion the top and bottom layers can move is dependent on how far each polymer can give or slide before the combined force overcomes the shear force. When this occurs the skin will slide on top of the foam producing greater shear forces or the polymers will break. Under prolonged shearing force the material eventually fatigues and fails. This results in material compression or more commonly cracks and tears.

Likewise, with athletic equipment, for example socks, the problem of blistering after extended periods of activity is well known. When an athlete endures high physical stress, the magnitude and frequency of the skin rubbing against the inner surface of a sock or other high-impact area, is increased when compared to normal daily activity. Thus, the blistering caused by such shearing forces is a common ailment of many athletes. The ability of a sock to prevent this blistering has been heretofore limited to different materials and weaves, principally for the purpose of providing cushioning. Providing a sock with reduced shear forces is unknown. The same is true of gloves and points of contact with various padding, such as sheets, pillow cases, and other bed linens.

A number of prior art patents have attempted to solve the problem without success. U.S. Pat. No. 5,615,418 issued Apr. 1, 1997 discloses a low-friction textile comprising at least three layers used, for example, in manufacturing a sock. A “low friction” material such as vinyl, satin, or mylar is sandwiched between layers of more traditional garment materials in order to reduce abrasion of the wearer's skin.

U.S. Pat. No. 5,918,317 issued Jul. 6, 1999 discloses a single layer material woven with a low friction fluoropolymer thread, for example Teflon®, predominating its exterior surface. When used in, for example, a sock, the entire exterior surface of the garment is slippery, thereby preventing or eliminating abrasion of the wearer's skin. There are obvious concerns with regard to the safety of highly slippery socks.

U.S. Pat. No. 6,108,820 issued Aug. 29, 2000 discloses the material of the '317 patent used in a bi-layered fabric with the low friction surfaces being are placed adjacent to one another.

U.S. Pat. Nos. 6,061,829 issued May 16, 2000 and 6,143,368 issued Nov. 7, 2000 disclose fibers that are dependant on a chemical coating to impart a low friction quality.

U.S. Pat. Nos. 4,615,188 issued Oct. 7, 1986 and 4,843,844 issued Jul. 4, 1989 disclose two-ply athletic socks having a mating surface of one ply composed primarily of a low friction material such as polypropylene yarn.

### SUMMARY OF THE INVENTION

The present invention is a material including a novel weave and multiple layers oriented in a manner that permits the layers to move or glide in relation to one another. The novel weave and orientation of the material impart its low friction qualities. No inner layer is required. Shear forces are absorbed by the material itself and not transmitted to the skin of the user, thereby reducing or eliminating the likelihood of skin trauma. In contrast to previously known materials, the present invention is not dependent on the thickness of the material or the chemical properties of the polymer to allow for the motion to be taken up within the material. It is therefore possible to produce a material that is much thinner while more greatly reducing shear forces. The oriented fabric found in the present invention is designed to greatly reduce these shear forces. In tests, the coefficient of friction is so low that the shear forces are virtually eliminated. Thus, the potential for blister formation and ulcer formation is greatly reduced. The reduced-friction fabric system can be placed in strategic positions within a shoe or sock to reduce the risk of blister formation. Socks made of this material would greatly reduce blistering on the foot when engaged in the high stress conditions athletes often endure. Blistering on the foot is common when running. The cause is friction when the foot slides against the inner surface of the sock. A sock with its sole coated with the present invention would prevent or minimize such friction as the two layers of the present invention would move across each other instead of the foot sliding across the inner surface of the sock. In the shoe, the regions, which are most likely to develop blisters and calluses are around the heel, across the ball of the foot, and over the tips and tops of the toes.

Although this is an important breakthrough for all athletic individuals, or those that do a great deal of walking and running, this invention also greatly benefits individuals suffering with neuropathy. Peripheral sensory neuropathy reduces a person's ability to feel their feet. Consequently, they are not aware when a blister forms, or progresses to the point of ulceration, until blood is observed in a sock or on the floor. These individuals do not have the ability to detect when their skin has been injured. As a result, they continue to carry on with their normal activities until the breakdown of skin is so severe that they are at risk for deep infections.

Reduced friction cloth would greatly reduce the risk of ulcers in people with a peripheral neuropathy from diseases like diabetes. More importantly, it would help in the healing process by controlling the pathologic mechanical forces causing ulcers, and diminishing the injury to newly forming skin, which is extremely fragile. Once an ulcer is closed, it would help the area to remain closed, by controlling these dangerous shear forces.

Reduced friction cloth could also be utilized in quadriplegic and hemiplegic patients who are at risk for pressure sores due to prolonged sitting while possessing a neuropathy. These patients must be continually repositioning themselves to avoid prolonged pressure in one area. Often times when they reposition themselves their garments become entangled thereby unknowingly increasing the pressure. Reduced friction cloth could be produced or applied into their garments decreasing the occurrence of this.

Additionally, the present invention would be valuable on wound dressing devices. Plastic surgeons and those treating burns and ulcers require frictionless bandaging systems to reduce the level of mechanical stress on the superficial skin structures. Standard dressings, which adhere to a wound, can easily disrupt new skin grafts or cause deeper injuries to slowly healing wounds by shearing the layers of skin. A frictionless system would allow the patient greater mobility by allowing movement, even adjacent to bony prominences and joints.

These and other objects, advantages, and novel features of the present invention will become apparent when considered with the teachings contained in the detailed disclosure along with the accompanying drawings.

### BRIEF DESCRIPTION OF THE DRAWINGS

FIG. 1 is an exploded schematic showing the top of the material with shear force applied thereto with the bottom surface fixed;

FIG. 2 is a cross sectional view the fabric of the present invention with the layers placed in orthogonal relationship;

FIG. 3 is an enlarged planar schematic view of the fabric of the present invention showing an over four under one pattern weave;

FIG. 4 is a schematic representation of the shear forces applied in an X side by side and Y front to back relationship with respect to the force collection plate on which testing was undertaken;

FIG. 5 is an exploded schematic representation of the two layers of fabric with the weave oriented and attached to form the low friction fabric;

FIG. 6 is a graph showing shear reactive force applied across the heel for a period of time with the same fiber under two different alignments;

FIG. 7 is a graph showing coefficients of friction for various fiber orientations;

FIG. 8 is both an exploded and top plan view of a shoe insert or insole and an exploded bottom plan view of a sock; and

FIG. 9 is a bottom plan view of a bandage using the low friction fabric of the present invention.

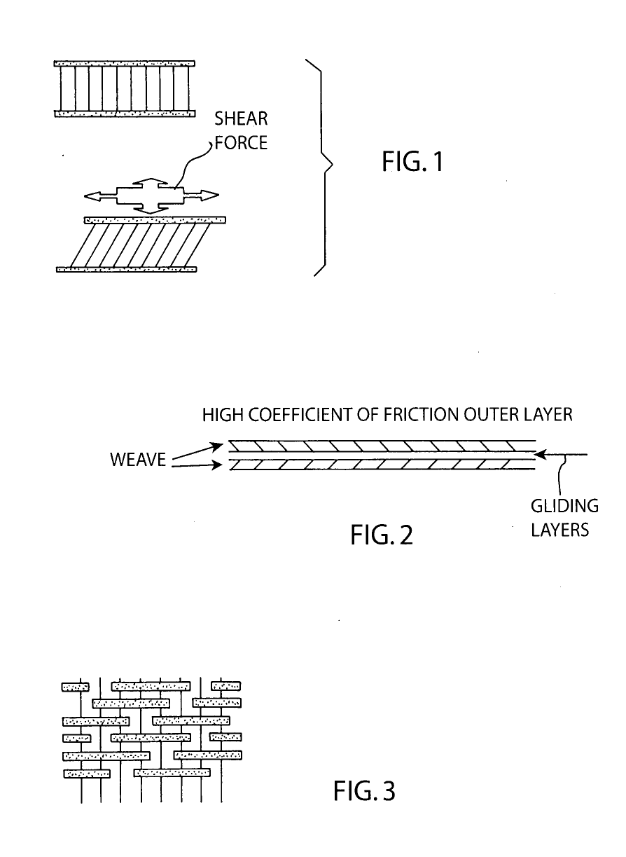

### DESCRIPTION OF THE INVENTION

### Definitions

As used in this invention:

Anisotropy is the property of being directionally dependent, as opposed to isotropy, which means hogeneity in all directions. It is a difference in a physical property for some material when measured along different axes. With regard to the instant invention, it is measured in changing coefficients of friction in response to the application of shear forces.

Orthogonal is used here synonymously with “perpendicular”, meaning at right angles.

The present invention is directed towards two fabric layers positioned at an angle to each other to create a reduced friction cloth. A woven fabric is composed of two yarns, interlocking from two directions. When viewing a piece of cloth, the fibers that are running the length of the cloth are known as the warp yarns and the fibers running perpendicular to these are known as the weft yarns. The long sides of the fabric are the selvage ends. These finished ends are made by the weft yarns turning around to weave back through the warp.

There are different patterns to weaving and different combinations of yarn types to make a specific fabric. An oxford shirt for example uses the over, under, over, under etc. pattern for the weft yarns, with the warp and weft yarns of the same material. If this weave were examined closely it would appear the same in from all directions.

The inventive reduced friction cloth uses a different weave and two different types of yarn to achieve its smooth side and its rough side. The material used includes two polyester fibers, although other material compositions would be suitable, including acetate, acrylic, cotton, elastic latex fibers (e.g., Lastex®), linen, nylon, rayon, silk, velvet, Spancex®, wool, or combinations thereof. Substitution of other materials is obvious to those skilled in the art. The warp yarn is a very straight yarn and the weft yarn being a low twist yarn. The weft travels over four and under one in the weaving pattern, although again, different weaves are possible and the use of other weaves would be obvious to those skilled in the art. This weave allows for much more surface area of the filling yarn to be exposed. The orientation of this surface is what produces the different properties. When the material is placed back upon itself or aligned so the weft fibers are parallel to each other the material has a high coefficient of friction. Coefficients of friction are correspondingly reduced as the angular orientation of the fibers approaches 90 degrees between layers. Maximum reduction in friction was measured at an orientation of substantially 90 degrees.

Two layers of such a weave fabric are combined to produce the reduced friction cloth. By adjusting the angle at which the layers are related, an increase or decrease of the friction between the layers can be achieved. Tests indicate that a maximum friction is achieved when the weaves are oriented in parallel, and a minimum friction is achieved when the weaves are orthogonal.

### Test Results

### EXAMPLE I

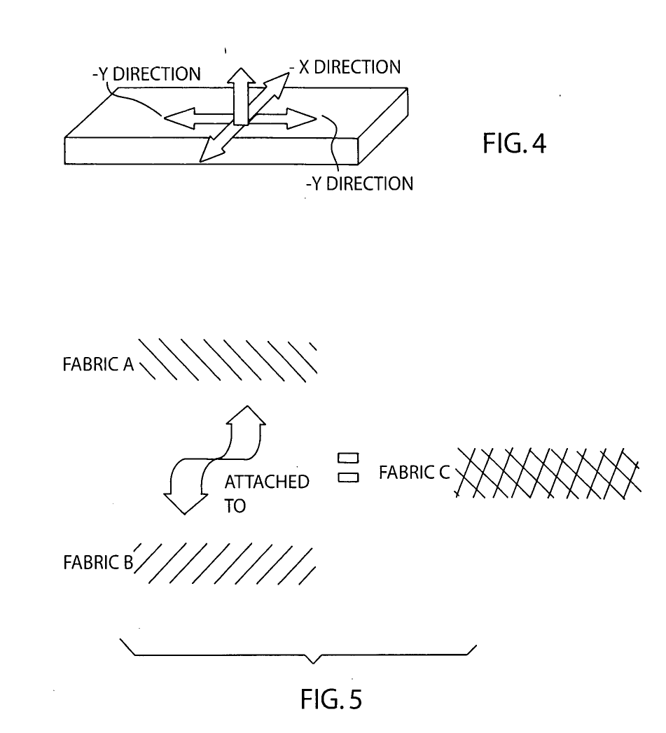

The cloth was placed between the heel and a Bertec force plate sampling at 120 Hz. The two components of the shear force is separated into an ±X medial to lateral (side to side) and an ±Y anterior to posterior (front to back) component with respect to the force collection plate. The positive and negative values only indicate direction of the force with respect to the center of the plate as seen in FIG. 4.

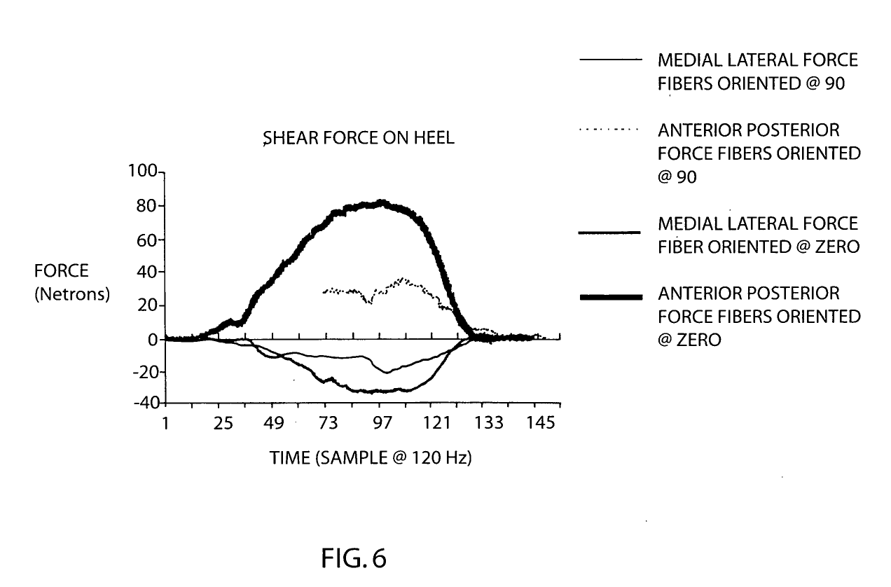

The graph of FIG. 6 shows the shear reactive force being applied across the heel for a period of time with the same fiber rendered in two different alignments. Fibers oriented at zero are aligned while those indicated at 90 are orthogonal to each other. The plot shows movement about the Z axis in the plane formed by X (medial to lateral) and Y (anterior to posterior) axes. Note that shear forces are minimized when fibers are oriented orthogonally.

### EXAMPLE II

Using a TMI (Testing Machines Inc.) Model 32-06 Slip Friction Tester was calibrated and was running in an environment of 72 degrees Fahrenheit at 40% humidity. The following test was performed:

An 8.5-cm by 33-cm sample of the fiber was fixed to the bed of the test unit. A 6.5-cm by 6.5-cm sample of the fiber was then fixed to the sled of the test unit with the fibers oriented in the same direction as the fibers on the test bed of the unit. This was designated as a 0 (zero) degree orientation. A test for static and dynamic coefficients of friction was then performed according to the ASTM D 1894 protocol. The static measurement is a reflection of the larger frictional forces during the initiation of motion while the kinetic measurement reflects the friction occurring once the sled was already moving. Thirty tests were performed using the same samples for each test.

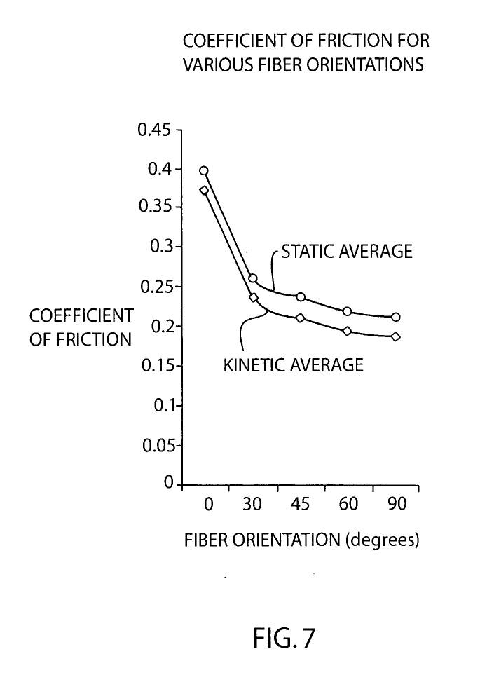

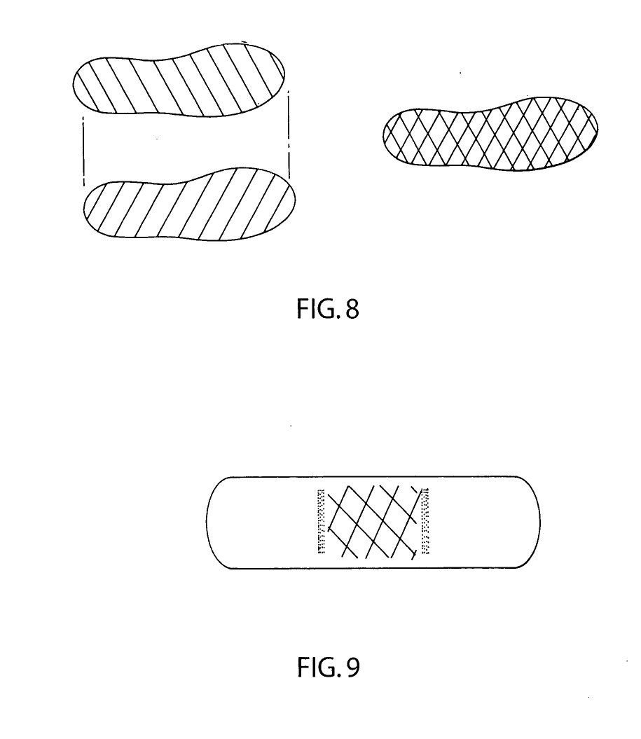

The original sample on the sled was then replaced with a sample of the same fiber type with the direction of the fibers oriented at 30, 45, 60, and 90 degrees to the sample on the bed of the machine. This was designated as a 30, 45,60 and 90-degree orientation respectively. Using the same test as described above, 115 additional tests were performed. Below are the statistical results. See also FIG. 7. Coefficients of friction are minimized at a fiber orientation of 90 degrees (i.e., orthogonal), corresponding with minimized shear forces. Optimal results are achieved with orthogonally oriented fibers. However, as shown in the experimental data below, any anisotropic weave (i.e., any orientation other than 0 or 180 degrees) corresponds with a reduction in shear forces.

| Static | 0 degrees | 30 degrees | 45 degrees | 60 degrees | 90 degrees |
|---|---|---|---|---|---|
| N | 30 | 30 | 25 | 30 | 30 |
| Average | 0.3971 | 0.259 | 0.2353 | 0.217 | 0.2097 |
| St dev | 0.0082 | 0.0082 | 0.0136 | 0.0081 | 0.0106 |
| Min | 0.382 | 0.245 | 0.213 | 0.202 | 0.192 |
| Max | 0.425 | 0.28 | 0.263 | 0.231 | 0.233 |

| Kinetic | 0 degrees | 30 degrees | 45 degrees | 60 degrees | 90 degrees |
|---|---|---|---|---|---|
| N | 30 | 30 | 25 | 30 | 30 |
| Average | 0.3729 | 0.235 | 0.2096 | 0.193 | 0.1849 |
| St dev | 0.009 | 0.0063 | 0.0116 | 0.0036 | 0.007 |
| Min | 0.351 | 0.222 | 0.191 | 0.186 | 0.176 |
| Max | 0.396 | 0.25 | 0.236 | 0.199 | 0.207 |

| % difference | Static | Kinetic |
|---|---|---|
| 0 vs 30 | −36.93 | −34.73 |
| 0 vs 45 | −40.74 | −43.8 |
| 0 vs 60 | −45.35 | −48.25 |
| 0 vs 90 | −47.19 | −50.42 |

Some other applications of the invention include, but are not limited to: In the area of medicine, the product could be used in making:
Bandages and/or pads applied to areas of the body to help avoid friction.
Socks for diabetics or related podiatric ailments.
Bed coverings such as sheets, pillowcases and bed linens for bedridden patients. In the area of recreation, the product could be used in making:
Pads that protect shoulders, elbows, thighs, knees and other body parts.
Innersole of a shoe or as an insert that can be added to a shoe.
Bicycle seat or a covering for an existing bicycle seat.
Undergarment athletic clothing such as underwear, shorts, shirts and the like.
Outer garment athletic clothing such as shirts, jerseys, pants.
Car seats or travel cushions.

The principles, preferred embodiments and modes of operation of the present invention have been described in the foregoing specification. However, the invention should not be construed as limited to the particular embodiments which have been described above. Instead, the embodiments described here should be regarded as illustrative rather than restrictive. Variations and changes may be made by others without departing from the scope of the present invention as defined by the following claims:

What is claimed is:

1. A low friction fabric comprising a first layer of woven material including a first upper and first lower woven surface and a second layer having a second upper and second lower woven surface, said second layer being in contact with said first lower surface of said first layer having a weave of a first orientation contacting said second upper surface of said second layer, said second layer having a weave oriented from 30 to 90 degrees relative to said first layer.

2. The low friction fabric of claim 1 wherein said fabric is constructed as a sock.

3. The low friction fabric of claim 1 wherein said fabric is a bandage.

4. The low friction fabric of claim 1 wherein said fabric is constructed as a shoe insert.

5. The low friction fabric of claim 1 wherein said fabric is constructed as a woven material taken from a group consisting of a sheet, a pillowcase and a bed linen.

6. The low friction fabric as claimed in claim 1 wherein said woven fabric is an over four under one weave.

7. The low friction fabric as claimed in claim 1 wherein said second layer is oriented substantially 30 degrees relative to said first layer.

8. The low friction fabric as claimed in claim 1 wherein said second layer is oriented substantially 45 degrees relative to said first layer.

9. The low friction fabric as claimed in claim 1 wherein said second layer is oriented substantially 60 degrees relative to said first layer.

10. The low friction fabric as claimed in claim 1 wherein said second layer is oriented substantially 90 degrees relative to said first layer.

11. The low friction fabric as claimed in claim 1 wherein said fabric is constructed of fibers chosen from the group consisting of polyester, acetate, acrylic, cotton, elastic latex fibers (e.g., Lastex®), linen, nylon, rayon, silk, velvet, Spancex®, wool, and combinations thereof.

12. A low friction fabric having a smooth side and a rough side comprising a first layer of woven fibers with a first upper and first lower woven surface attached to and adjacent a second layer of woven fibers, said second layer having a second upper and second lower surface, said first and second woven layers each comprising a weave with a straight yarn in the warp of the weave pattern and a low twist yarn in the weft of the weave pattern, said first lower surface of said first layer being in contact with the said second upper surface of said second layer with the weft yarn of said layers oriented at from 30 to 90 degrees angle relative to one another.

13. The low friction fabric of claim 12 wherein said fabric is constructed as a sock.

14. The low friction fabric of claim 12 wherein said fabric is a bandage.

15. The low friction fabric of claim 12 wherein said fabric is constructed as a shoe insert.

16. The low friction fabric of claim 12 wherein said fabric is constructed as a sheet, pillowcase, or other bed linen.

17. A low friction fabric as claimed in claim 12 wherein said woven fabric is an over four under one weave.

18. The low friction fabric as claimed in claim 12 wherein said second layer is oriented substantially 30 degrees relative to said first layer.

19. The low friction fabric as claimed in claim 12 wherein said second layer is oriented substantially 45 degrees relative to said first layer.

20. The low friction fabric as claimed in claim 12 wherein said second layer is oriented substantially 60 degrees relative to said first layer.

21. The low friction fabric as claimed in claim 12 wherein said second layer is oriented substantially 90 degrees relative to said first layer.

22. A low friction fabric comprising a first layer of woven fibers having an over four under one weave with a first upper and first lower surface attached to a second layer of woven fibers having an over four under one weave with a second upper and second lower surface, said first and second layers comprising a straight yarn in the warp of the weave pattern and a low twist yarn in the weft of the weave pattern, said first lower surface of said first layer being in contact with said second upper surface of said second layer with the weft yarn of said layers being oriented from 30 to 90 degrees relative to one another.

23. A low friction fabric comprising a first layer of woven material with a first upper and first lower woven surface attached to a second layer of woven material of the same weave, said second layer having a second upper and second lower woven surface, said fabric layers being in contact and said first lower surface of said first layer having a weave of a first orientation contacting the entire said second upper surface of said second layer, said second layer having a weave with a second orientation substantially orthogonal to the weave of said first layer to reduce the coefficient of friction between the layers.
